(19) United States
(12) Patent Application Publication
(10) Pub. No.: US 2024/0312019 A1
(43) Pub. Date: Sep. 19, 2024

(54) MEDICAL IMAGE PROCESSING APPARATUS, METHOD FOR OPERATING MEDICAL IMAGE PROCESSING APPARATUS, AND NON-TRANSITORY COMPUTER READABLE MEDIUM

(71) Applicant: FUJIFILM Corporation, Tokyo (JP)

(72) Inventor: Katsuyuki HIGA, Kanagawa (JP)

(73) Assignee: FUJIFILM Corporation, Tokyo (JP)

(21) Appl. No.: 18/676,391

(22) Filed: May 28, 2024

Related U.S. Application Data
(63) Continuation of application No. PCT/JP2022/034598, filed on Sep. 15, 2022.

(30) Foreign Application Priority Data
Nov. 30, 2021 (JP) 2021-194538

(51) Int. Cl. G06T 7/00; G16H 40/63

(52) U.S. Cl. CPC G06T 7/0016; G16H 40/63; G06T 2200/24; G06T 2207/20092; G06T 2207/30096

(57) ABSTRACT

A medical image processing apparatus performs a detection process of detecting a photographic subject from each medical image of a plurality of time-series medical images, and performs a post-detection process whose details are predetermined in accordance with the detected photographic subject. The post-detection process includes at least a process of providing a notification about the detected photographic subject to a user. In response to receipt of a specific instruction from the user after the notification has been provided, an operation mode is set to a disabled mode in which the post-detection process is disabled in a preset disabled period.

### CROSS-REFERENCE TO RELATED APPLICATIONS

This application is a Continuation of PCT International Application No. PCT/JP2022/034598 filed on 15 Sep. 2022, which claims priority under 35 U.S.C § 119(a) to Japanese Patent Application No. 2021-194538 filed on 30 Nov. 2021. The above application is hereby expressly incorporated by reference, in its entirety, into the present application.

### BACKGROUND OF THE INVENTION

### 1. Field of the Invention

The present invention relates to a medical image processing apparatus, a method for operating the medical image processing apparatus, and a non-transitory computer readable medium.

### 2. Description of the Related Art

In the medical field, an image detection process of detecting a photographic subject such as a lesion is performed using medical images acquired by various modalities, such as an endoscope, a computed tomography (CT) apparatus, or magnetic resonance imaging (MRI), to acquire diagnosis assistance information for assisting a doctor in making a diagnosis. In recent years, various methods for acquiring target information through an image detection process using a machine learning technique have been developed.

In an image detection process, it may be impossible to favorably perform the detection process for various reasons. Thus, efforts are made to acquire more appropriate information through an image detection process. For example, there is known an endoscope system including a notifying unit that determines an operation for a photographic subject in a medical image, sets a recognizing unit to be enabled or disabled by using a determination result, and provides a notification about an enabled or disabled state of the recognizing unit in an examination using an endoscope or the like (WO2020/036224A, corresponding to US2021/0177248A1). With this endoscope system, it is possible to prevent, when a medical image for which an image recognition process can function has not been acquired, a recognition result obtained by the recognizing unit for the medical image from being erroneously recognized as an appropriate recognition result.

### SUMMARY OF THE INVENTION

When appropriate information is not acquired in an image detection process, a cancellation mechanism that disables the image detection process may be used. When the cancellation mechanism is activated and the image detection process is disabled, if the image detection process is continuously suspended for a long time thereafter, diagnosis assistance information cannot be acquired during the duration. On the other hand, when the duration of suspension of the image detection process is short, it may be necessary to activate the cancellation mechanism many times after the duration ends, which is troublesome.

An object of the present invention is to provide a medical image processing apparatus, a method for operating the medical image processing apparatus, and a non-transitory computer readable medium that are capable of flexibly controlling a detection-related process in accordance with a user's desire.

A medical image processing apparatus according to the present invention includes a processor. The processor is configured to acquire a plurality of time-series medical images; perform a detection process of detecting a photographic subject from each medical image of the plurality of medical images; perform, in response to the photographic subject being detected, a post-detection process whose details are predetermined in accordance with the detected photographic subject, the post-detection process including at least a process of providing a notification about the detected photographic subject to a user; and set, in response to a specific instruction being provided from the user after the notification has been provided, an operation mode to a disabled mode in which the post-detection process for at least the detected photographic subject is disabled in a preset disabled period.

Preferably, the detection process is at least one of a lesion detection process of detecting a lesion, a treatment tool detection process of detecting a treatment tool, or an area detection process of detecting an area.

Preferably, the lesion detection process is a process of detecting the lesion on a type-by-type basis, and the treatment tool detection process is a process of detecting the treatment tool on a type-by-type basis.

Preferably, the post-detection process includes a process of recording a result of the detection process in a recording unit.

Preferably, the post-detection process includes a process of displaying, on a display, an option predetermined in accordance with the detected photographic subject.

Preferably, the processor is configured to receive input from the user, and determine, based on the input, whether the specific instruction has been provided.

Preferably, the processor is configured to determine that the specific instruction has been provided, in response to the input being continuously performed for a preset continuous-input determination period or more in an input reception period during which the input is received.

Preferably, the processor is configured to determine that the specific instruction has been provided, in response to the input being continuously performed for a preset continuous-input determination period or more in a period during which the post-detection process for the photographic subject is disabled.

Preferably, the processor is configured to determine that the specific instruction has been provided, in response to the input being performed at least once in an input reception period during which the input is received.

Preferably, the input reception period is a period during which the post-detection process is performed.

Preferably, the processor is configured to determine that the specific instruction has been provided, in response to the input being performed a preset number of times or more in a preset multiple-input determination period.

Preferably, the processor is configured to disable, in the disabled mode, the post-detection process for the lesion and/or a plurality of types of the treatment tool.

Preferably, the processor is configured to, when the post-detection process includes a plurality of processes, disable, in the disabled mode, at least one of the processes included in the post-detection process.

Preferably, the processor is configured to, in response to detecting the legion, disable, in the disabled mode, the post-detection process related to the lesion detection process.

Preferably, the processor is configured to, in response to detecting the treatment tool, disable, in the disabled mode, the post-detection process related to the treatment tool detection process.

Preferably, the processor is configured to, in response to detecting the area, disable, in the disabled mode, the post-detection process related to the area detection process.

Preferably, the processor is configured to disable, in the disabled mode, the detection process for the detected photographic subject.

Preferably, the processor is configured to disable, in the disabled mode, the detection process for the lesion and/or a plurality of types of the treatment tool.

Preferably, the processor is configured to, when the detection process includes a plurality of processes, disable, in the disabled mode, at least one of the processes included in the detection process.

Preferably, the processor is configured to set the disabled period to be a period from a time point at which the specific instruction is provided to a time point at which a specific cancellation condition is satisfied.

Preferably, the cancellation condition is that a photographic subject different from the detected photographic subject is detected in the medical image.

Preferably, the processor is configured to calculate a degree of similarity indicating a degree to which the plurality of medical images are similar to each other, and the cancellation condition is that the degree of similarity between the plurality of medical images acquired most recently is lower than or equal to a preset value.

Preferably, the processor is configured to provide a notification to the user in response to receiving the specific instruction from the user, in a case of setting the disabled mode, or in a case of cancelling setting of the disabled mode.

A method for operating a medical image processing apparatus according to the present invention includes a step of acquiring a plurality of time-series medical images; a step of performing a detection process of detecting a photographic subject from each medical image of the plurality of medical images; a step of performing, in response to the photographic subject being detected, a post-detection process whose details are predetermined in accordance with the detected photographic subject, the post-detection process including at least a process of providing a notification about the detected photographic subject to a user; and a step of setting, in response to a specific instruction being provided from the user after the notification has been provided, an operation mode to a disabled mode in which the post-detection process for at least the detected photographic subject is disabled in a preset disabled period.

A non-transitory computer readable medium according to the present invention is for storing a computer-executable program that causes a computer to execute a function of acquiring a plurality of time-series medical images; a function of performing a detection process of detecting a photographic subject from each medical image of the plurality of medical images; a function of performing, in response to the photographic subject being detected, a post-detection process whose details are predetermined in accordance with the detected photographic subject, the post-detection process including at least a process of providing a notification about the detected photographic subject to a user; and a function of setting, in response to a specific instruction being provided from the user after the notification has been provided, an operation mode to a disabled mode in which the post-detection process for at least the detected photographic subject is disabled in a preset disabled period.

According to the present invention, it is possible to flexibly control a detection-related process in accordance with a user's desire.

### BRIEF DESCRIPTION OF THE DRAWINGS

FIG. 1 is a block diagram illustrating the functions of a medical image processing apparatus;

FIG. 2 is a block diagram describing the configuration of the medical image processing apparatus;

FIG. 3 is a block diagram illustrating the functions of a detection processing unit;

FIG. 4 is a block diagram illustrating the functions of a lesion detecting unit, a treatment tool detecting unit, and an area detecting unit;

FIG. 5 is an explanatory diagram describing a detection process in which a treatment tool detector detects a treatment tool, and a post-detection process in which a notification about a detection result is provided;

FIG. 6 is an explanatory diagram describing a detection process in which the treatment tool detector detects a treatment tool, and a post-detection process in which a detection result is recorded together with an endoscopic image;

FIG. 7 is an explanatory diagram describing a post-detection process of displaying options;

FIG. 8 is an explanatory diagram describing a case where, in a detection process in which the treatment tool detector detects a treatment tool and a post-detection process in which a notification about a detection result is provided, the detection process is erroneous;

FIG. 9 is an explanatory diagram of a case where a method for determining a specific instruction is a method using a continuous-input determination period;

FIG. 10 is an explanatory diagram of a case where activation of a cancellation mechanism and shift to a disabled mode are successively performed;

FIG. 11 is an explanatory diagram of a case where a method for determining a specific instruction is a method in which input is performed at least once in an input reception period;

FIG. 12 is an explanatory diagram of a case where a method for determining a specific instruction is a method using a multiple-input determination period;

FIG. 13A is an explanatory diagram describing a case where a detection result indication of erroneous detection is not displayed and an indicator is displayed only for a predetermined period in a disabled mode;

FIG. 13B is an explanatory diagram describing a case where a detection result indication of erroneous detection is continuously displayed and an indicator is displayed only for a predetermined period in a disabled mode;

FIG. 14A is an explanatory diagram describing a case where a detection process or the like of forceps, which is a detected photographic subject, is not performed in a disabled mode;

FIG. 14B is an explanatory diagram describing a case where a detection process or the like of forceps, which is a detected photographic subject, is not performed but a detection process of a snare, which is other than the detected photographic subject, is performed;

FIG. 15A is an explanatory diagram describing a case where a detection process or the like of a treatment tool, which is a detected photographic subject, is not performed in a disabled mode;

FIG. 15B is an explanatory diagram describing a case where a detection process or the like of a treatment tool, which is a detected photographic subject, is not performed but a detection process of a lesion, which is other than the detected photographic subject, is performed;

FIG. 16 is an explanatory diagram describing that a disabled mode is cancelled in response to a cancellation condition for the disabled mode being satisfied;

FIG. 17 is an explanatory diagram describing a case where a cancellation condition for a disabled mode is satisfied when a photographic subject different from a detected photographic subject is detected;

FIG. 18 is a block diagram illustrating the functions of the medical image processing apparatus including a degree-of-similarity calculating unit;

FIG. 19 is an explanatory diagram describing a case where a cancellation condition for a disabled mode is satisfied when the degree of similarity between endoscopic images is low; and

FIG. 20 is a flowchart illustrating an example of a flow of a process of the medical image processing apparatus.

### DESCRIPTION OF THE PREFERRED EMBODIMENTS

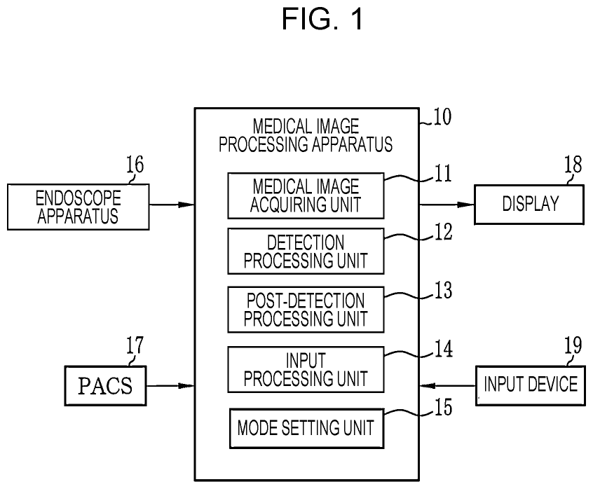

An example of a basic configuration of the present invention will be described. As illustrated in FIG. 1, a medical image processing apparatus 10 includes a medical image acquiring unit 11, a detection processing unit 12, a post-detection processing unit 13, an input processing unit 14, and a mode setting unit 15. The medical image processing apparatus 10 is connected to an endoscope apparatus 16, various modalities (not illustrated) for X-ray examinations or the like, an examination information system (not illustrated) such as a radiology information system (RIS) or an endoscope information system, an apparatus capable of outputting medical image data such as a picture archiving and communication system (PACS, a medical image management system) 17, a display device such as a display 18, and/or an input device 19 such as a keyboard (not illustrated), a touch panel of the display 18, or a foot switch.

The medical image processing apparatus 10 performs a detection process of detecting a photographic subject from each medical image of a plurality of time-series medical images acquired from the endoscope apparatus 16 or the like. The medical images are, for example, medical images handled by the PACS 17, and are images mainly acquired in an examination. Specifically, the medical images are X-ray images acquired in an X-ray examination, MRI images acquired in an MR examination, CT images acquired in a CT examination, endoscopic images acquired in an endoscopic examination, or ultrasound images acquired in an ultrasound examination.

The photographic subject to be detected is a photographic subject to be detected in each detection process of detecting a specific photographic subject among photographic subjects in a medical image, and is, for example, any of various lesions, any of various treatment tools, or any of various areas. Thus, the detection process includes a lesion detection process of detecting a lesion, a treatment tool detection process of detecting a treatment tool, an area detection process of detecting each area, and so forth. At least one of these detection processes is performed, and two or more of these detection processes may be performed.

The detection process is performed by a diagnosis assistance learning model that is constructed by performing learning on a machine learning algorithm. The diagnosis assistance learning model is a learning model that has been subjected to learning, adjustment, and so forth so as to output, in response to input of a medical image, information about a detected photographic subject, which is a result of an intended detection process (hereinafter referred to as a detection result).

A post-detection process is performed on the detection result acquired in the detection process. In the post-detection process, the details of the process are predetermined in accordance with the detected photographic subject. The post-detection process having the predetermined details is performed on the detected photographic subject.

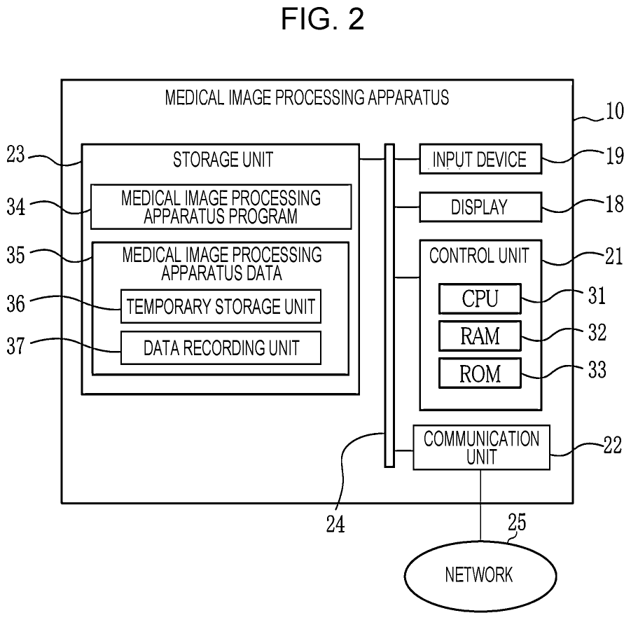

The post-detection process includes a notification process of providing a notification about the detected photographic subject to the user. The post-detection process includes, in addition to the notification process, a recording process of recording a detection result in a data recording unit 37 (see FIG. 2), an option display process of displaying options for the detected photographic subject on the display 18, and so forth. In the option display process, the user makes a selection regarding the detected photographic subject from among the displayed options. The recorded detection result or the selected content about the detected photographic subject is used to create a report or the like.

The notification is provided by, for example, display on the display 18. Thus, when a photographic subject has been detected, information about the detected photographic subject, which is a detection result, such as the name of the photographic subject, is displayed on the display 18 and thus the user is notified of the information. During an examination, an acquired medical image is displayed on the display 18, and thus the medical image and a detection result are displayed on the display 18. Thus, the user is able to know the detection result by viewing the display 18 during observation in the examination. The user uses the detection result displayed on the display 18 as diagnosis assistance information for the examination, diagnosis, or the like.

After the notification, the user may view the medical image and the detection result and determine that the detection result is erroneous. In this case, the user is able to provide a specific instruction to set the operation mode of the medical image processing apparatus 10 to a disabled mode. The details of the disabled mode can be preset.

The medical image processing apparatus 10 sets the disabled mode in which a post-detection process is disabled, in response to receipt of a specific instruction from a user. The disabled mode is an operation mode in which a post-detection process for at least a detected photographic subject is disabled. When a post-detection process is disabled in the disabled mode, a post-detection process predetermined in accordance with the detected photographic subject and including at least a notification process is not performed. The disabled mode is applied during a preset period. The medical image processing apparatus 10 cancels the disabled mode in response to a cancellation condition for cancelling the disabled mode being satisfied.

The user is able to flexibly apply the disabled mode for disabling a post-detection process by setting the start timing of the disabled mode, the details of the disabled mode, or the like. In addition, the disabled mode can be cancelled, for example, upon a cancellation condition for the disabled mode being satisfied, and the cancellation condition can be set by the user.

In an examination or treatment in which a medical image is captured, a report is created after the examination or treatment. For example, a diagnosis assistance function may be used for assistance in creating a report of an endoscopic diagnosis using an endoscope. The diagnosis assistance function automatically detects the details of treatment, a treatment tool, an observation area, or the like by using so-called artificial intelligence (AI) or the like, such as a diagnosis assistance learning model based on machine learning, records a detection result, and creates a report by using the recorded detection result.

Here, if the detection result obtained by AI or the like is erroneous, the user may perform a cancellation operation of activating a cancellation mechanism for a detection process or the like by using the input device 19, such as a foot switch, and disable the detection process or the like in a predetermined period. However, if erroneous detections by AI occur consecutively, it may be troublesome for the user to repeatedly perform the cancellation operation. On the other hand, if the user once performs a cancellation operation and keeps a detection process or the like disabled for a long time, the detection process or the like is not performed during the time, and it is impossible to effectively use the diagnosis assistance function.

The medical image processing apparatus 10 is capable of presetting the details of a post-detection process indicating how to use a detection result obtained by AI. When an erroneous detection or the like occurs, the medical image processing apparatus 10 shifts the operation mode to a disabled mode in which the post-detection process is disabled, in response to a specific instruction provided by the user. In the disabled mode, a post-detection process or the like is disabled. The duration of the disabled mode until the disabled mode is cancelled can be preset in accordance with various cancellation conditions. The disabled mode can be continuously performed under a predetermined condition. Thus, even if erroneous detections by AI occur consecutively, the user does not need to repeatedly perform a cancellation operation, and a trouble involved in the cancellation operation can be avoided.

In the case of setting the duration of the disabled mode, cancellation of the disabled mode can be set in a preset cancellation condition. As a result of appropriately setting cancellation of the disabled mode, the detection process by AI can be automatically restarted when the detection process is to be restarted. Thus, the diagnosis assistance function can be effectively used while a trouble involved in a cancellation operation is avoided. In this specification, the terms “cancel” and “disable” are used in similar meaning. Disabling of each process means that the effect targeted by the process does not appear, and does not mean that no process is performed.

As described above, the medical image processing apparatus 10 is capable of flexibly controlling a process related to detection by AI or the like in accordance with a user's desire, and is capable of performing various processes related to an examination using a medical image, such as creation of a report or use of a detection result, more accurately and efficiently.

An embodiment of the medical image processing apparatus 10 of the present invention will be described. As illustrated in FIG. 2, the medical image processing apparatus 10 according to the present embodiment has a hardware configuration of a computer in which the input device 19 serving as an input device, the display 18 mainly serving as an output device, a control unit 21, a communication unit 22, and a storage unit 23 are electrically connected to each other via a data bus 24.

The input device 19 is an input device such as a keyboard, a mouse, a touch panel of the display 18, a foot switch, or a scope button of the endoscope apparatus 16. The computer constituting the medical image processing apparatus 10 receives input of various instructions from the input device 19. The display 18 is a kind of output device. The display 18 displays various operation screens in accordance with operations of the input device 19. Each operation screen is equipped with an operation function using a graphical user interface (GUI). The computer constituting the medical image processing apparatus 10 is capable of receiving input from a user through the operation screen.

The control unit 21 includes a central processing unit (CPU) 31 serving as a processor, a random access memory (RAM) 32, a read only memory (ROM) 33, and the like. The CPU 31 loads a program stored in the storage unit 23 or the like to the RAM 32 or the ROM 33 and executes a process in accordance with the program, thereby centrally controlling individual components of the computer. The communication unit 22 is a network interface that controls transmission of various pieces of information via a network 25. The RAM 32 or the ROM 33 may have the function of the storage unit 23.

The storage unit 23 is an example of a memory and is, for example, a hard disk drive, a solid state drive, or a disk array including a plurality of hard disk drives or the like that is built in the computer constituting the medical image processing apparatus 10 or is connected thereto through a cable or a network. The storage unit 23 stores a control program, various application programs, various data to be used for these programs, display data of various operation screens accompanying these programs, and so forth.

The storage unit 23 according to the present embodiment stores various data such as a medical image processing apparatus program 34 and medical image processing apparatus data 35. The medical image processing apparatus program 34 or the medical image processing apparatus data 35 is a program or data for implementing various functions of the medical image processing apparatus 10. The medical image processing apparatus program 34 and the medical image processing apparatus data 35 implement the functions of the medical image processing apparatus 10. The medical image processing apparatus data 35 includes a temporary storage unit 36 and the data recording unit 37, and data or the like temporarily stored by the medical image processing apparatus program 34 is also stored therein.

The computer constituting the medical image processing apparatus 10 may be a purpose-designed apparatus, a general-purpose server apparatus, a personal computer (PC), or the like. It is sufficient that the functions of the medical image processing apparatus 10 be exhibited. The computer or a part of the computer may also serve as an apparatus having another function, or the functions of the medical image processing apparatus 10 may be incorporated into an endoscope management system or the like.

The medical image processing apparatus 10 according to the present embodiment is a processor apparatus. In the medical image processing apparatus 10, a program related to medical image processing is stored in the storage unit 23, which is a program memory. In the medical image processing apparatus 10, a program in the program memory is operated by the control unit 21 constituted by a processor or the like, and thereby the functions of the medical image acquiring unit 11, the detection processing unit 12, the post-detection processing unit 13, the input processing unit 14, and the mode setting unit 15 are implemented (see FIG. 1).

The medical image acquiring unit 11 acquires a plurality of time-series medical images from an apparatus capable of outputting medical images. The medical images are images mainly acquired in an examination, and may be images acquired in real time during the examination or images acquired from stored medical images captured during the examination. The plurality of time-series medical images may be a plurality of medical images acquired in one examination.

In the present embodiment, endoscopic images are acquired in real time in an endoscopic examination using the endoscope apparatus 16. Thus, the medical image acquiring unit 11 acquires a plurality of time-series endoscopic images. An endoscopic image is a kind of medical image and is an image acquired by imaging a subject by using an endoscope included in the endoscope apparatus 16. Hereinafter, a description will be given of a case where endoscopic images are used as medical images. Endoscopic images are images based on individual frame images captured by the endoscope apparatus 16 in a preset number of frames and mainly mean still images. The display 18 displays, for an endoscopic examination, a moving image composed of a plurality of time-series frame images that are sequentially displayed.

The detection processing unit 12 performs a detection process of detecting a specific photographic subject from each endoscopic image of the plurality of endoscopic images acquired by the medical image acquiring unit 11. In the present embodiment, the detection process is sequentially performed on each endoscopic image of a plurality of time-series endoscopic images, normally in the order in which the endoscopic images have been acquired.

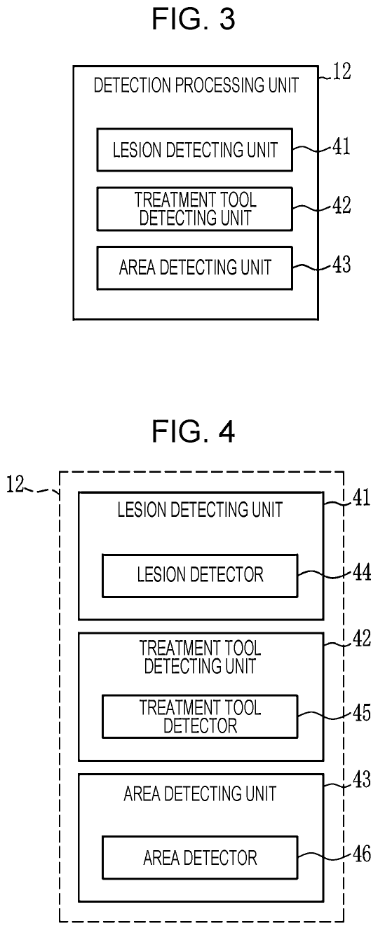

As illustrated in FIG. 3, the detection processing unit 12 includes a lesion detecting unit 41, a treatment tool detecting unit 42, and an area detecting unit 43. Based on an acquired endoscopic image, the lesion detecting unit 41 performs a detection process of detecting a region of interest such as a lesion, the treatment tool detecting unit 42 performs a detection process of detecting a treatment tool of any of various types, and the area detecting unit 43 performs a detection process of detecting an area. The photographic subject detected by the lesion detecting unit 41 is a lesion, the photographic subject detected by the treatment tool detecting unit 42 is a treatment tool, and the photographic subject detected by the area detecting unit 43 is an area. These detection processes detect photographic subjects of different categories, such as a lesion, a treatment tool, and an area.

The lesion detecting unit 41 may perform, as a lesion detection process, a process of detecting a lesion on a type-by-type basis. The photographic subject detected in the case of detecting a lesion on a type-by-type basis may be of any type related to a lesion, and may be, for example, a tumor or a non-tumor, the degree of progress of various types, the score of a diagnosis criterion of various types, or the like.

Similarly, the treatment tool detecting unit 42 may perform, as a treatment tool detection process, a process of detecting a treatment tool on a type-by-type basis. The photographic subject detected in the case of detecting a treatment tool on a type-by-type basis may be of any type related to a treatment tool, and may be, for example, a type based on the name of a treatment tool, a type based on the purpose of a treatment tool, a type based on a procedure, or the like. In a type based on the name of a treatment tool or a type based on the purpose of a treatment tool, the photographic subject is a snare, forceps, a knife, or the like. In a type based on a procedure, the photographic subject may be a treatment tool of a type for use in polypectomy, cold polypectomy, endoscopic mucosal resection (EMR), or the like.

The detection processing unit 12 may perform a measurement process, a classification process, or the like as a detection process. For example, it is possible to perform a measurement process, a classification process, or the like of detecting a value of biological information such as oxygen saturation. In this case, the detection processing unit 12 includes a measurement unit (not illustrated) or a classification unit (not illustrated). In the case of detecting a value of biological information, a detection process of detecting a region or the like of a living body having an oxygen saturation within a specific value range may be performed as a measurement process, and a detection process of classifying the region or the like of the living body for each value range of oxygen saturation may be performed as a classification process.

In a detection process, the position, the size, the distance from the distal end portion of the endoscope, or the like of a lesion, a treatment tool, an area, or the like may be determined, or the lesion, the treatment tool, the area, or the like may be subjected to discrimination or the like. In the case of performing discrimination or the like in the detection process, a photographic subject having a discrimination result is detected. These detection processes may be performed as one detection process or a combination of a plurality of detection processes.

As illustrated in FIG. 4, each detecting unit included in the detection processing unit 12 can include one or more detectors suitable for a specific photographic subject to be detected. The detector or detectors may be provided outside the medical image processing apparatus 10. The lesion detecting unit 41 includes a lesion detector 44. The lesion detector 44 is a diagnosis assistance learning model that detects, based on an acquired endoscopic image, a lesion of a photographic subject. In response to an endoscopic image being input and when a photographic subject in the endoscopic image includes a lesion, the lesion detector 44 generates a detection result including at least the region of the lesion in the photographic subject. The detection result may include, in addition to the region of the lesion, various pieces of information acquired by training the lesion detector 44, such as the type or category of the lesion, such as a redness, a tumor, or a non-tumor, or the stage or degree of progress of a specific lesion. The detection result may also include stored information or the like that can be acquired from another apparatus. When the photographic subject in the endoscopic image does not include a lesion, a detection result indicating “no lesion” may be generated.

The treatment tool detecting unit 42 includes a treatment tool detector 45. The treatment tool detecting unit 42 is a diagnosis assistance learning model that detects, based on an acquired endoscopic image, a treatment tool of any of various types in the endoscopic image. In response to an endoscopic image being input and when a photographic subject in the endoscopic image includes a treatment tool of any of various types, the treatment tool detector 45 generates a detection result including the name of the treatment tool. The detection result includes, in addition to the name of the treatment tool, various pieces of information acquired by training the treatment tool detector 45, such as the manufacturer name, the model number, the date of manufacture, the date of start of use, or the like of the treatment tool. The detection result may include stored information or the like that can be acquired from another apparatus. When the photographic subject in the endoscopic image does not include a treatment tool of any of various types, a detection result indicating “no treatment tool” may be generated.

The area detecting unit 43 includes an area detector 46. The area detecting unit 43 is a diagnosis assistance learning model that detects, based on an acquired endoscopic image, an area of a photographic subject in the endoscopic image. In response to an endoscopic image being input, the area detector 46 generates a detection result including the name of an area of a photographic subject in the endoscopic image. The detection result includes, in addition to the name of the area, various pieces of information acquired by training the area detector 46, such as the position or orientation of the endoscope at the time of capturing the endoscopic image. The detection result may also include stored information or the like that can be acquired from another apparatus. When the photographic subject in the endoscopic image does not include a specific area, a detection result indicating “no area detected” may be generated.

The various detectors including the lesion detector 44, the treatment tool detector 45, and the area detector 46 are specifically diagnosis assistance learning models constructed by using a machine learning algorithm, and are learning models that are capable of, in response to an endoscopic image 51 being input to each detector, detecting a specific photographic subject which is a detection target included in the endoscopic image 51 and outputting information about a detection result as an objective variable. The various detectors have been trained in advance by using a machine learning algorithm and an initial image data set for the various detectors constituted by the endoscopic image 51 including a specific photographic subject and correct answer data of the name, position, or the like of the specific photographic subject, and parameters or the like have been adjusted, so that information about a detection result of the specific photographic subject included in the endoscopic image 51 can be output as an objective variable.

A machine learning algorithm used for the various detectors may be any algorithm mainly used for supervised learning. It is preferable to use an algorithm that is to output a favorable inference result as an objective variable in a detection process using an image. For example, it is preferable to use a multilayer neural network or a convolutional neural network, and it is preferable to use a method called deep learning. The diagnosis assistance learning model may employ a technique commonly used to improve the performance of a learning model, for example, to increase the accuracy of detecting a specific photographic subject or increase the detection speed, such as processing of the endoscopic image 51 serving as an input image, use of a plurality of learning models, or a combination with unsupervised learning. Instead of being constituted by a detector based on machine learning, the detection processing unit 12 may be configured to calculate a feature quantity from an endoscopic image and perform detection or the like by using the calculated feature quantity.

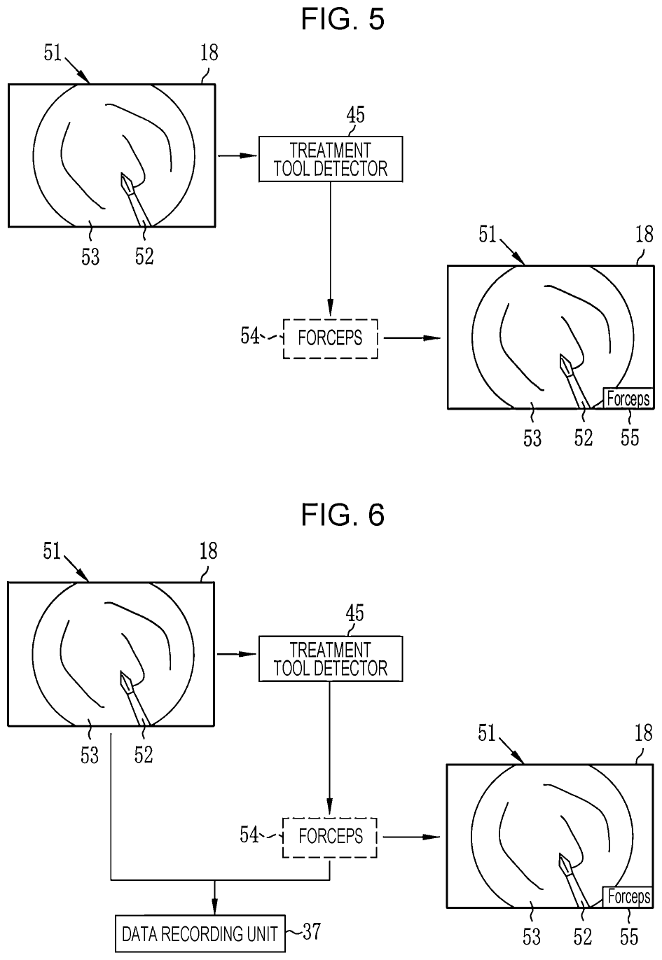

As illustrated in FIG. 5, during an examination, the endoscopic image 51 is displayed in the form of a moving image on the display 18. The treatment tool detector 45 performs a detection process of detecting a treatment tool of any of various types as a photographic subject to be detected from the endoscopic image 51. When the endoscopic image 51 has, as photographic subjects, forceps 52 and a lumen wall 53 which is a subject, a detected photographic subjects is a treatment tool of any of various types. When the forceps 52, which is a kind of treatment tool, is detected by the detection processing unit 12, a treatment tool name “forceps” is generated as a detection result 54.

The post-detection processing unit 13 performs a post-detection process. The post-detection process includes various processes that use the detection result 54. In the post-detection process, the details of the process are predetermined in accordance with a detected photographic subject. The post-detection process includes at least a notification process of providing a notification about the detected photographic subject to a user. The notification process may be notification related to the detected photographic subject, and includes notifying a user of the name of the detected treatment tool, and notifying a user of the name of a treatment performed at the time of detection in accordance with the detected photographic subject. The post-detection process is performed in response to a photographic subject being detected. In the present embodiment, the detected photographic subject is a treatment tool of any of various types, and thus the post-detection process is performed in response to any treatment tool being detected.

As described above, the post-detection process includes, in addition to a notification process of providing a notification about the detection result 54 or the like, a recording process of recording the detection result 54 in a recording unit, an option display process of displaying, on the display 18, options predetermined for a detected photographic subject, and so forth. In the post-detection process, performing of a plurality of processes selected from among various processes may be defined as the details of the process.

In the option display process, options for selecting contents to be described in a report among various pieces of information related to the detected photographic subject are displayed, for example. When a treatment tool name has been detected, options of a procedure using the detected treatment tool are displayed. When an area has been detected, options of an area name related to the detected area are displayed. The options may be displayed in the form of text or may be displayed in the form of a schematic diagram or the like. By selecting a procedure, an area name, or the like from among the options, the user is able to cause the procedure using the detected treatment tool or the area name to be automatically described in the report.

In various processes included in the post-detection process, means of the processes are preset. For example, in a notification process, notification means is display on the display 18. In a recording process, recording means is recording in the data recording unit 37. In an option display process, option display means is display on the display 18.

The post-detection process may be performed in combination with a manual process. For example, when a lesion is detected in a detection process, the name of an area may be manually input by a user selecting the area from a schematic diagram of an examination target, for example. After that, a treatment tool may be detected in a detection process and options related to the treatment tool may be displayed in a post-detection process. As a result of combining a manual process and a post-detection process as appropriate, operation suitable for various situations can be performed.

In the present embodiment, after the detection process, the detection result 54 indicating that the forceps 52 have been detected is displayed as a detection result indication 55 on the display 18 in a notification process. The detection result indication 55 notifies the user that the medical image processing apparatus 10 has detected the forceps 52 in the detection process.

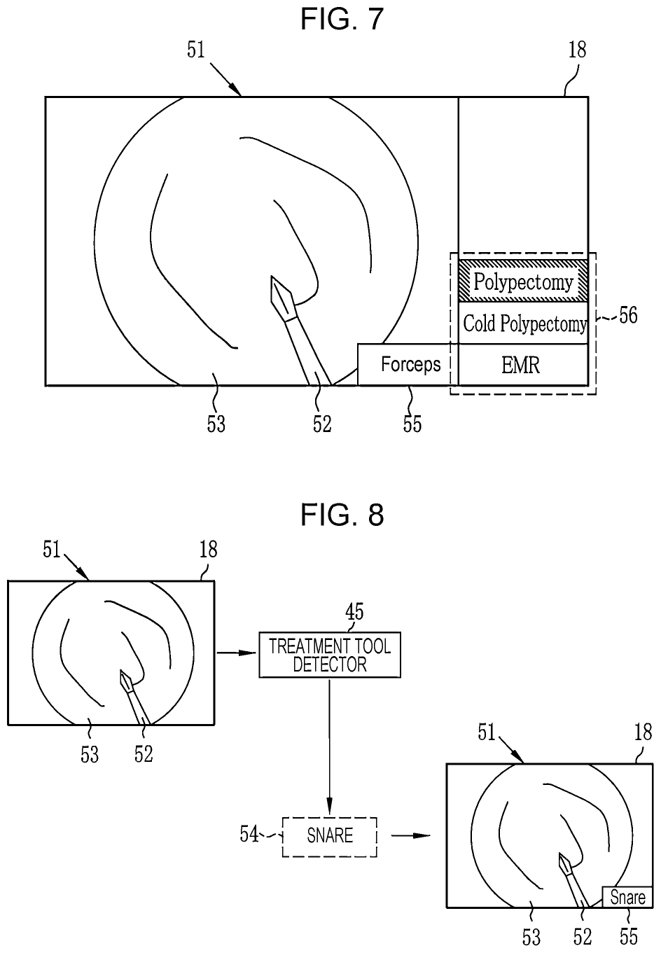

As illustrated in FIG. 6, in a recording process, a treatment tool name “forceps” is recorded as the detection result 54 in the data recording unit 37 in association with the endoscopic image 51 in which the treatment tool has been detected. As illustrated in FIG. 7, the options of a procedure associated with the treatment tool name “forceps” are “Polypectomy”, “Cold Polypectomy”, and “EMR”. Thus, in an option display process, the options, which are the names of these procedures using “forceps” indicated by the detection result 54, are displayed as an option indication 56 on the display 18. As a result of a procedure name to be described in an examination report being selected from the option indication 56, the procedure name is confirmed and recorded. In FIG. 7, “Polypectomy” has been selected and confirmed. After the notification has been provided or the options have been displayed, the user continues the endoscopic examination without providing a specific instruction if the treatment tool has been properly detected in the detection process.

In an endoscopic examination, a detection process may generate an erroneous detection result. For example, as illustrated in FIG. 8, “snare” may be generated as the detection result 54 when the forceps 52 are used and depicted in the endoscopic image 51. As in the case where the detection result 54 correctly indicates the forceps 52, the detection result 54 “snare” is displayed as the detection result indication 55 on the display 18 in a notification process. The detection result indication 55 notifies the user that the detection result 54 obtained in the detection process performed by the medical image processing apparatus 10 based on the endoscopic image 51 is “snare”. In a recording process, the treatment tool name “snare”, which is the detection result 54, is recorded in the data recording unit 37 in association with the endoscopic image 51 in which the treatment tool has been detected.

After the notification, the user is able to determine whether detection has been correctly performed or erroneously performed in the detection process, by viewing the endoscopic image 51 and the detection result indication 55 displayed on the display 18. When the user determines that erroneous detection has occurred in the detection process, the user sets the operation mode of the medical image processing apparatus 10 to the disabled mode so that erroneous display, recording, or the like is not performed any longer. Accordingly, a post-detection process or the like can be disabled. Disabling of a post-detection process or the like prevents unnecessary effort, such as correcting of an erroneous record later. To set the disabled mode, a specific instruction is provided to the medical image processing apparatus 10. A method for providing the specific instruction is preset, and thus the specific instruction is identified.

The specific instruction may be based on input by the user. The input processing unit 14 receives input from the user and determines whether the specific instruction has been provided. To determine the specific instruction based on input from the user, the medical image processing apparatus 10 includes the input processing unit 14 that receives input from the user. The user performs input by using the input device 19, and the input processing unit 14 determines, based on the input from the user, whether the specific instruction has been provided.

In an input method using the input device 19, when the input device 19 is a keyboard, a mouse, a touch panel of the display 18, or a scope switch of the endoscope apparatus 16, for example, selection, click, or touch of a specific button or the like is regarded as input. When the input device 19 is a foot switch, a press of the foot switch is regarded as input. When a plurality of scope switches or foot switches are provided, a specific scope switch or foot switch is preset.

In the present embodiment, the scope switch or foot switch is a so-called momentary switch that is in an ON state while being operated by pressing or the like. The momentary switch is in an OFF state while being released after pressing has finished. Depending on cases, various switches, such as a so-called alternate switch that is maintained in an ON state, may be used. The alternate switch is switched between an ON state and an OFF state by pressing. When the alternate switch is employed, it is assumed that input is being performed in an ON state.

In the case of determining input from the user, the input processing unit 14 employs a determination method selected by the user from among a plurality of determination methods. One of the plurality of determination methods is a method in which input is continuously performed for a preset continuous-input determination period or more in an input reception period.

The input reception period is a period during which input from the user is received in order for the input processing unit 14 to determine the input from the user, and can be preset. For example, the input reception period is a period during which a post-detection process is performed. As described above, the post-detection process includes at least a notification process among the notification process, the recording process, and the option display process. Thus, a period during which a notification process such as displaying of the detection result indication 55 is performed is referred to as an input reception period. The period during which the detection result indication 55 is displayed is a period during which detection result indications 55 based on a plurality of detection results 54 sequentially generated from a plurality of time-series endoscopic images 51 are sequentially displayed.

The continuous-input determination period is a period for determining whether input is a specific instruction. When input is continuously performed beyond the period, it is determined that a specific instruction has been provided. “Input is continuously performed” means that the ON state of the switch for performing input continues. For example, when input is pressing of a foot switch, a period during which the foot switch is kept pressed is a period during which input is continuously performed.

The continuous-input determination period is preset and is, for example, 3 seconds. The continuous-input determination period starts upon input being performed. In a case where input is performed, a continuous-input determination period is started, and the continuation of the input is cancelled before the preset continuous-input determination period elapses, the continuous-input determination period ends. After that, upon input being performed, the continuous-input determination period restarts.

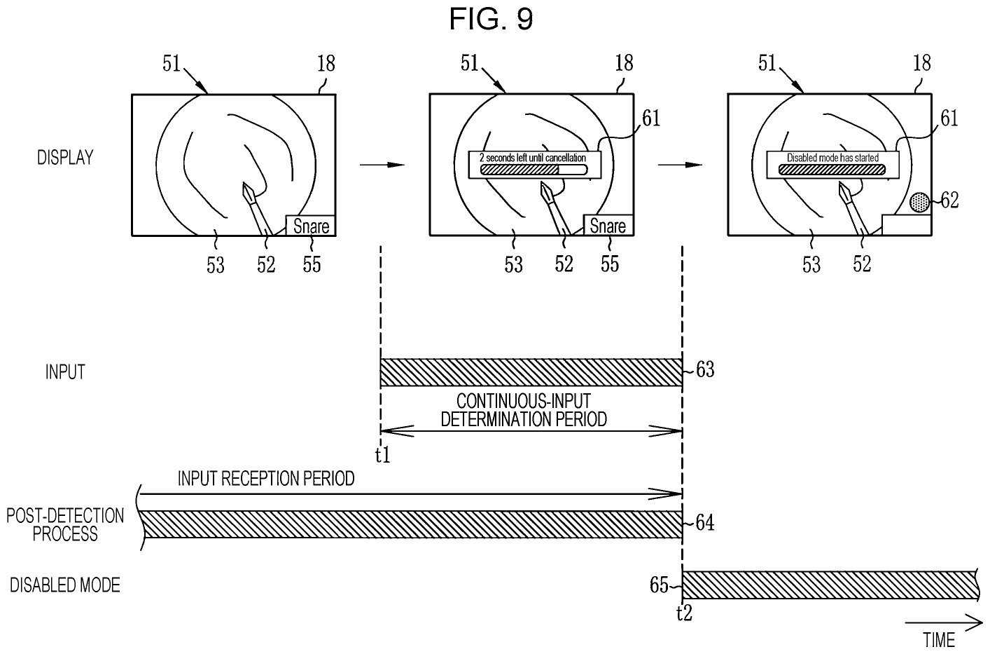

As illustrated in FIG. 9, the user views the detection result indication 55 displayed on the display 18, determines that erroneous detection has been performed in the detection process, and provides a specific instruction to switch the operation mode to the disabled mode. In FIG. 9, time elapses from left to right, and display images displayed on the display 18 are illustrated in a chronological order on the right side of “display”. On the right side of “input”, “post-detection process”, and “disabled mode”, periods during which the respective processes are executed are indicated in a bar graph manner. Input is executed in an input period 63. The post-detection process is performed in a post-detection process period 64. In the present embodiment, the post-detection process period 64 is the same as the input reception period. The disabled mode is executed in a disabled mode period 65.

At time t1, the user starts input by pressing the foot switch in the input reception period during which the detection result indication 55 is displayed. After time t1 at which pressing of the foot switch is started, an indicator 61 is displayed on the display 18 and indicates the remaining input period 63 during which the foot switch is to be kept pressed to cause the input period 63 to become longer than or equal to the continuous-input determination period and cause a specific instruction to be provided. For example, the indicator 61 formed of text “2 seconds left until cancellation” and a figure that changes as time elapses, indicates a period until the input period 63 becomes longer than or equal to the continuous-input determination period.

When the input period 63 during which pressing of the foot switch is performed as input continues for a period longer than a preset continuous-input determination period, the input processing unit 14 determines that a specific instruction has been provided. The continuous-input determination period is a period from time t1 to time t2. At time t2 at which the input period 63 reaches the continuous-input determination period, a specific instruction is provided. In response to the specific instruction being provided, an instruction is provided from the input processing unit 14 to the mode setting unit 15, and at time t2, the operation mode of the medical image processing apparatus 10 is switched to the disabled mode by the mode setting unit 15.

In a disabled period, a post-detection process for a detected photographic subject is disabled. Thus, in the present embodiment, in the disabled mode, a detection process is performed, but a notification process, a recording process, or an option display process included in a post-detection process is not performed. In the present embodiment, a post-detection process is disabled in the disabled mode, and thus the detection result indication 55 is not displayed in the disabled mode. As will be described below, a setting can be made so as not to perform a detection process in the disabled mode. The post-detection process is a process whose details are determined in accordance with the detected photographic subject. Thus, when a detection process is not performed, there is no detected photographic subject and thus the post-detection process is not performed.

For the visibility of the endoscopic image 51, at the time of switching to the disabled mode, the indicator 61 may indicate that the disabled mode has started for a preset period of time, for example, several seconds, and then the indicator 61 may be hidden. In the present embodiment, as illustrated in FIG. 9, the indicator 61 indicates text “disabled mode has started. The disabled mode continues for a preset period. While the disabled mode continues, a disabled mode mark 62 may stay lit up so that the user is able to grasp that the disabled mode is active at a glance.

As a result of providing a specific instruction by using the continuous-input determination period, it is possible to prevent erroneous pressing of the foot switch, use of the foot switch to provide another instruction, or the like from being determined as a specific instruction against the intention of the user. In addition, by viewing the indicator 61, the user is able to know, for example, how much time is necessary until a specific instruction is executed, and is also able to stop the shift to the disabled mode by stopping input before the continuous-input determination period elapses.

A cancellation mechanism of disabling a detection process or a post-detection process in a short period and the shift to the disabled mode may be employed in combination. Thus, a specific instruction may be that input is continuously performed for a preset continuous-input reception period or more in a period during which a post-detection process for a photographic subject is disabled.

For various reasons, for example, during treatment using the forceps 52, the treatment tool detector 45 may erroneously generate a treatment tool name other than the forceps 52 as the detection result 54. In preparation for such a case, the medical image processing apparatus 10 includes a cancellation mechanism that disables recording or the like of a detection result in a short period, such as several seconds, like an existing medical image processing apparatus or the like that does not have a function of shifting to the disabled mode. The cancellation mechanism is activated by input using an input unit.

The cancellation mechanism is activated in response to an instruction different from the specific instruction, for example, in response to input being performed a preset number of times of input for the cancellation mechanism or more in a preset determination period for the cancellation mechanism. Specifically, the determination period for the cancellation mechanism starts from the time point of the first input, the determination period for the cancellation mechanism is 2 seconds, and the number of times of input for the cancellation mechanism is 3 or more, for example. Thus, the cancellation mechanism is activated in response to input for the cancellation mechanism being performed three times or more within 2 seconds, which is the determination period for the cancellation mechanism. After the activation of the cancellation mechanism has been confirmed, a detection process or a post-detection process is disabled for, for example, 3 seconds after the confirmation, and the disablement is automatically cancelled after 3 seconds have elapsed.

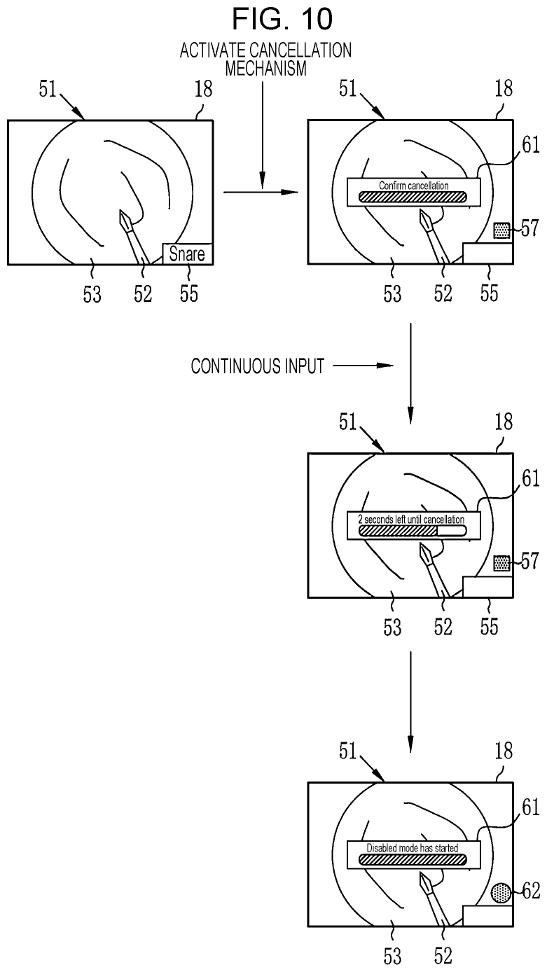

As illustrated in FIG. 10, the cancellation mechanism is activated by, for example, three inputs such as pressing of the foot switch, and the user is notified that the activation has been confirmed. The notification is provided by the indicator 61 on the display 18, a cancellation mechanism activation mark 57, or the like. As an input method for activating the cancellation mechanism, various input methods can be employed, such as continuous input, that is, long-pressing of the input device 19, as well as performing input the number of times of input for the cancellation mechanism or more in the determination period for the cancellation mechanism. Also in this case, it is preferable to activate the cancellation mechanism by using a determination method different from that in the continuous-input determination period for shifting to the disabled mode, so as be distinguished from the shift to the disabled mode.

As one of methods of employing the cancellation mechanism and the shift to the disabled mode in combination, switching to the disabled mode is performed by using the cancellation mechanism. For example, when the user wants a post-detection process to be continuously disabled after the activation of the cancellation mechanism has been confirmed, the user may perform input to provide a specific instruction subsequently to the input for activating the cancellation mechanism. As the input serving as a specific instruction, the above-described method using a continuous-input determination period in which continuous input (long press) is performed by using the input device 19, or the like is employed. The method for performing continuous input is similar to that described above. Text or the like such as “2 seconds left until cancellation” is displayed by the indicator 61. At the time of shift to the disabled mode, text such as “disabled mode has started” and a figure indicating the remaining time before shifting to the disabled mode are displayed.

Accordingly, in the cancellation mechanism, disablement is cancelled after the specific short period, but it is not necessary to perform input for activating the cancellation mechanism again when a detection process or a post-detection process is to be disabled again. Thus, in the medical image processing apparatus 10, switching to the disabled mode can be executed while the conventional cancellation mechanism is maintained. Thus, the user is able to use the medical image processing apparatus 10 without a sense of discomfort, and convenience is further increased.

One of the plurality of determination methods is a method in which input is performed at least once in an input reception period. In the present embodiment, the input reception period is the post-detection process period 64. Thus, when a touch of the touch panel, a press of the foot switch, or the like is performed at least once from the start of the post-detection process, that is, the start of notification about of the detection result 54, it is determined that a specific instruction has been provided. In this case, it is determined that input has been performed once when an ON state occurs in the input reception period regardless of the type of switch.

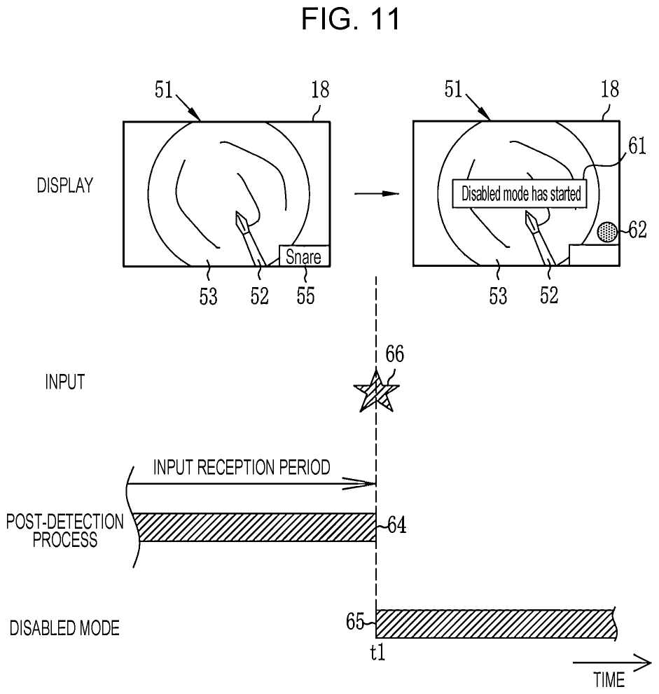

As illustrated in FIG. 11, the user views the detection result indication 55 displayed on the display 18, determines that erroneous detection has been performed in the detection process, and provides a specific instruction to switch the operation mode to the disabled mode. FIG. 11 is similar to FIG. 9 except for the method of determining input to be a specific instruction.

The user performs input of pressing the foot switch once at time t1. In FIG. 11, input 66 using the foot switch is indicated by a star-shaped mark. At time t1 at which the input 66 is performed by pressing the foot switch once, the input processing unit 14 determines that a specific instruction has been provided. The operation of the medical image processing apparatus 10 performed in response to the specific instruction is similar to that in the case of FIG. 9. In this way, the specific instruction is provided by performing input once, and thus the operation mode can be quickly switched.

One of the plurality of determination methods is a method in which input is performed a preset number of times or more in a preset multiple-input determination period. The multiple-input determination period is a period for determining whether input is a specific instruction. When input is performed a specific number of times or more within this period, it is determined that a specific instruction has been provided. “Input is performed once” is similar to the case in FIG. 11 and means that the switch for performing input has been brought into an ON state once. For example, when input is a press of the foot switch, it is assumed that one press of the foot switch corresponds to one input. Thus, in the present embodiment, input using the foot switch is determined to be input using a momentary switch. Thus, for the second input, the press of the foot switch is cancelled to set the foot switch to an OFF state, and then the foot switch is pressed again to set the foot switch to an ON state. The specific number of times is preset and is, for example, 3.

The multiple-input determination period is preset and is, for example, 3 seconds. The multiple-input determination period starts upon input being performed. When the first input has been performed, a multiple-input determination period has started, and input has been performed a specific number of times before the preset multiple-input determination period elapses, a specific instruction is executed. In the present embodiment, a momentary switch is employed. Thus, one press of the switch in a state where the switch has not been pressed corresponds to one input. Thus, in a case where input is performed three times or more within 3 seconds, which is the multiple-input determination period, this condition is satisfied as a result of a press and stop of the press being performed approximately three times within 3 seconds.

When input has been performed, a multiple-input determination period has started, and input has not been performed the specific number of times within the preset multiple-input determination period, the multiple-input determination period ends. After that, upon input being performed, the multiple-input determination period starts again.

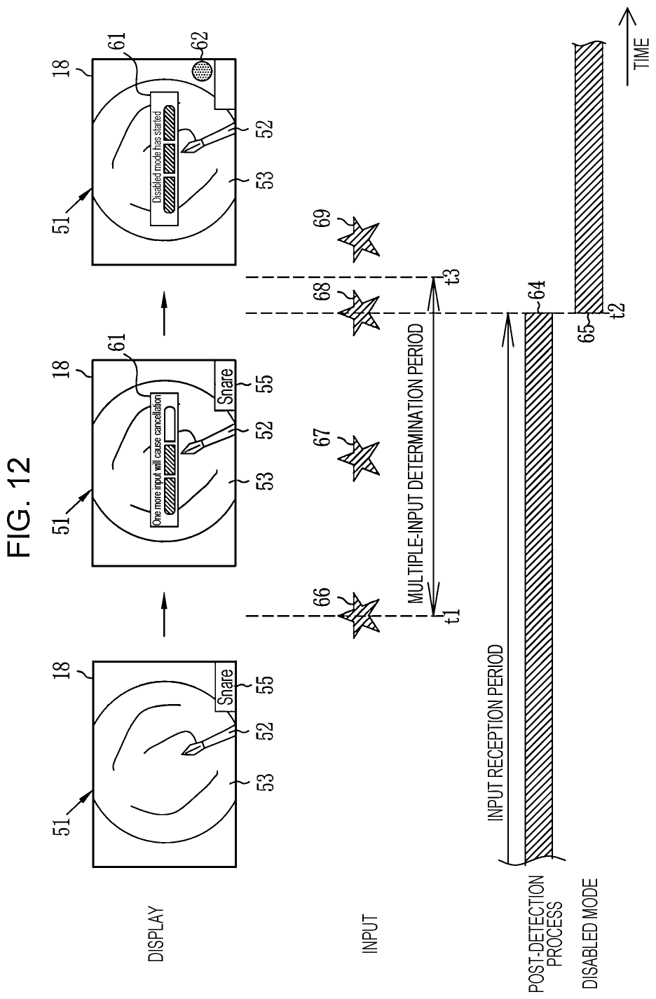

As illustrated in FIG. 12, by viewing the detection result indication 55 displayed on the display 18, the user provides a specific instruction to switch the operation mode to the disabled mode. FIG. 12 is similar to FIG. 9 except for a determination method of determining input to be a specific instruction.

By viewing the detection result indication 55, the user performs input of pressing the foot switch once at time t1 by the input 66. After time t1 at which the input 66 is performed, the indicator 61 is displayed on the display 18 and indicates the number of times remaining until the number of presses of the foot switch becomes the number of times of input or more. For example, after input 67, the text “one more input will cause cancellation” and figures whose color is changed by a press and which indicate that input has been performed twice are displayed, and thereby the number of times of input remaining until the number becomes the number of times of input or more is indicated. In accordance with the multiple-input determination period, when the multiple-input determination period, for example, 3 seconds, has elapsed from the first input 66, one of the figures whose color was changed first by a press is changed to an original color. Accordingly, the indicator 61 makes it possible to recognize that a specific instruction is provided by continuously pressing the foot switch in the multiple-input determination period.

Upon pressing of the foot switch being performed as input a number of times greater than the preset number of times of input in the multiple-input determination period, the input processing unit 14 determines that a specific instruction has been provided. The multiple-input determination period is a period from time t1 to time t3. Pressing of the foot switch is performed four times: input 66; input 67; input 68; and input 69. Time t2 at which the input 68, which is the third input, is performed is earlier than time t3. The specific instruction is provided at time t2 at which the input 68 is performed. The operation performed in response to the specific instruction is similar to that in the case of FIG. 9.

As a result of providing a specific instruction by using the multiple-input determination period, it is possible to prevent, when the foot switch is erroneously pressed or when the foot switch is used to provide another instruction, the press or use from being determined as a specific instruction. In addition, by viewing the indicator 61, the user is able to know how many inputs are further necessary to execute the specific instruction, and is able to stop the shift to the disabled mode by, for example, performing pressing a set number of times or less so as not to perform multiple inputs in the multiple-input determination period.

Preferably, in response to receipt of a specific instruction from the user, the input processing unit 14 notifies the user of the receipt. When the specific instruction has been received and the disabled mode is to be started, a notification indicating that the specific instruction has been received may be common to a notification indicating that the operation mode is the disabled mode.

As described above, a method for determining a specific instruction provided using input can be selected and set from among a plurality of determination methods by setting. These determination methods enable the user to provide a specific instruction by performing input using an easy method that does not hinder an examination. In addition, it is possible to select an appropriate determination method in accordance with use or the like and perform switching to the disabled mode.

The mode setting unit 15 sets the operation mode of the medical image processing apparatus 10 to the disabled mode, based on an instruction or the like from the input processing unit 14. The disabled mode is an operation mode in which a detection process or a post-detection process for at least a detected photographic subject is disabled in a preset period. The input processing unit 14 determines a specific instruction from the user. Upon determining that a specific instruction has been received from the user, the input processing unit 14 provides an instruction to set the operation mode of the medical image processing apparatus 10 to the disabled mode to the mode setting unit 15.

The details of the disabled mode are preset by setting. In the disabled mode, the mode setting unit 15 is capable of disabling a post-detection process for a detected photographic subject as described above. When a detection process is a lesion detection process of detecting a lesion on a type-by-type basis, a treatment tool detection process of detecting a treatment tool on a type-by-type basis, or the like, the mode setting unit 15 is capable of disabling, in accordance with the detection process, a post-detection process for a plurality of types of lesions and/or a plurality of types of treatment tools.

When the post-detection process includes a plurality of processes, the mode setting unit 15 is capable of disabling, in the disabled mode, at least one of the processes included in the post-detection process. Alternatively, all the processes included in the post-search post-detection process may be disabled. In the present embodiment, a detection process is performed, and a post-detection process using the detection result 54 is not performed. In the disabled mode, none of the notification process, the recording process, and the option display process that are set as the post-detection process is performed, but a specific process that is not included in the post-detection process can be performed on the detection result 54. In the disabled mode, among the notification process, the recording process, and the option display process that are set to be included in the post-detection process, the recording process and the option display process may be performed, with only the notification process not being performed, for example.

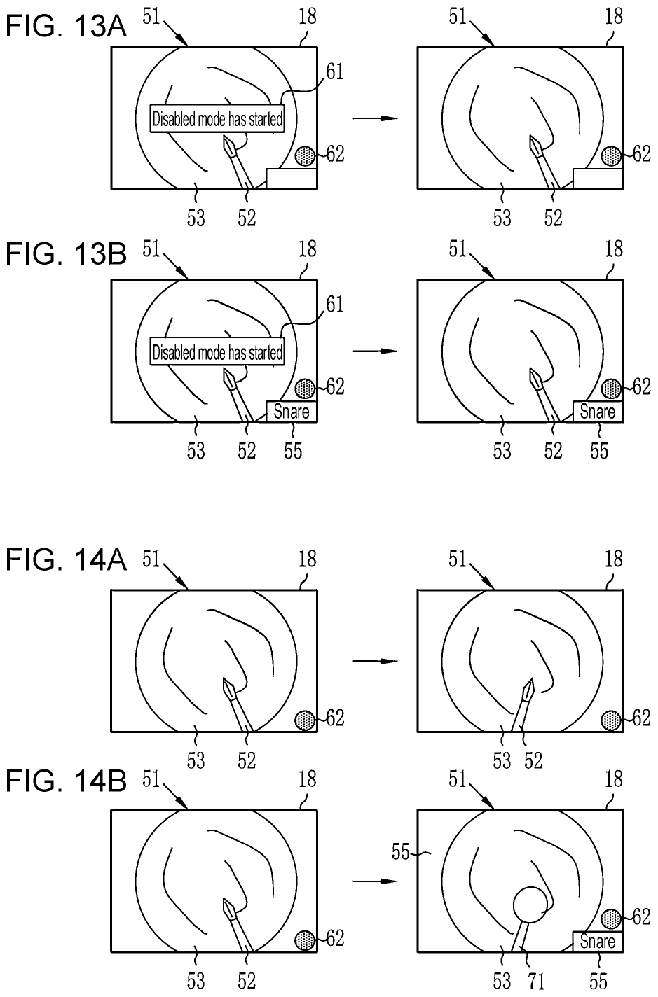

As illustrated in FIG. 13A, as a result of the disabled mode being set, “disabled mode has started” or the like is displayed on the indicator 61 for a predetermined period, such as first several seconds, after the shift from an operation mode other than the disabled mode to the disabled mode, so that the user is notified that the disabled mode has started. After the predetermined period has elapsed, the indicator 61 is hidden so as not to interfere with observation. In the disabled mode, it is preferable to constantly continue display on the display 18 or the like to provide a notification about the disabled mode. For example, the disabled mode mark 62 displayed on the display 18 may stay lit up while the disabled mode is set so that the user is notified that the operation mode is the disabled mode and is able to grasp the notification at a glance.

As illustrated in FIG. 13B, when the post-detection process is disabled, the last displayed detection result indication 55 may be kept displayed. The case illustrated in FIG. 13B is similar to the case in FIG. 13A except that the detection result indication 55 is kept displayed. As a result of keeping the detection result indication 55 displayed, the situation at the time of disabling can be grasped in the disabled mode, and information about the time to cancel the disabled mode can be provided to the user.

In the disabled mode, the mode setting unit 15 is capable of making a setting to disable a detection process for a detected photographic subject. When a detection process is not performed, there is no detected photographic subject, and thus a post-detection process is not performed. The details of the case where a post-detection process is not performed are similar to those described above. When a detection process is not performed, it is impossible to perform various processes using a detection result, whereas calculation resources used for the detection process are not necessary. Thus, it is possible to increase a processing speed, for example.

When the detection process is a lesion detection process of detecting a lesion on a type-by-type basis, a treatment tool detection process of detecting a treatment tool on a type-by-type basis, or the like, the mode setting unit 15 is capable of disabling, in accordance with the detection process, the detection process for a plurality of types of lesions and/or a plurality of types of treatment tools. When the detection process includes a plurality of processes, the mode setting unit 15 is capable of disabling, in the disabled mode, at least one of the processes included in the detection process. Alternatively, all the processes included in the detection process may be disabled. For example, when a setting is made to detect a plurality of photographic subjects of categories different from each other, such as a lesion, a treatment tool, or an area, in a detection process, the detection process for at least one of these categories can be disabled, or the detection process for photographic subjects of all the categories can be disabled.

Disabling of a detection process and disabling of a post-detection process in the disabled mode can be selected and set in advance. In the disabled mode, the details of the detection process and the details of the post-detection process can be combined, for example, the detection process for a photographic subject of a category is disabled and the detection process for a photographic subject of another category is enabled, and furthermore, a part of the post-detection process is disabled and another part of the post-detection process is enabled.

The color of the disabled mode mark 62 may be made different between a state in which the detection process is disabled and a state in which the post-detection process is disabled, for example, to notify the user of the state. Alternatively, the process that is disabled may be displayed to provide a notification using a figure such as a mark, text, or the like.

As a result of disabling a detection process or a post-detection process in the disabled mode, it is possible to prevent erroneous detection from continuing and erroneous display, recording, or the like from being performed any longer when erroneous detection is performed in the detection process. In addition, disabling of a detection process and disabling of a post-detection process are selectable, and thus it is possible to perform flexible handling with a setting in which a part of the detection process is performed but a part of the post-detection process, that is, display or recording, is not performed. In this case, the detection result 54 is not used in the post-detection process, but the detection result 54 can be used for another purpose. In the case of disabling the detection process, it is possible to achieve, for example, saving of calculation resources.

In the disabled mode, the mode setting unit 15 is capable of presetting, in accordance with a detection process, the photographic subject in a case where the detection process or the post-detection process is disabled, and is also capable of presetting the range of the photographic subject in detail.

For example, when the detection process is, for example, a detection process of detecting a treatment tool on a type-by-type basis, a detected photographic subject as a target of disabling may be a photographic subject of the same type as the detected photographic subject. For example, when the forceps 52 are detected, the type of the detected photographic subject is “forceps”, and a snare or the like, which is a photographic subject other than forceps, is not regarded as a target of disabling.

As illustrated in FIG. 14A, when the forceps 52 are erroneously detected as a snare, the detected photographic subject is the forceps 52, and thus a detection process or a post-detection process for the forceps 52 is disabled in the disabled mode. Thus, in the disabled mode, when the forceps 52 are included in the endoscopic image 51, a detection process or a post-detection process for the forceps 52 is not performed. Because the disabled mode in which the detection process or the like for the forceps 52 is disabled is continued, the disabled mode mark 62 is displayed on the display 18.

As illustrated in FIG. 14B, in the disabled mode in which a detection process or a post-detection process for the forceps 52 is disabled, when a detection process or a post-detection process for the forceps 52 is not performed and the endoscopic image 51 includes the snare 71, a detection process or a post-detection process for the snare 71 is performed in the detection process by the detection processing unit 12. In the detection process for the snare 71, for example, “snare” is generated as the detection result 54 and is displayed as the detection result indication 55 on the display 18. Because the disabled mode in which a detection process or the like for the forceps 52 is disabled is continued, the disabled mode mark 62 is displayed on the display 18.

In addition, for example, when the detection process is a detection process of not detecting a treatment tool on a type-by-type basis but detecting a treatment tool and another photographic subject while discriminating therebetween, the detected photographic subject as a target of disabling may be all treatment tools. For example, when the forceps 52 are detected, the category of the photographic subject including the forceps is treatment tool. Thus, a photographic subject as a target of disabling is a photographic subject of a treatment tool. When the forceps 52 are detected, the detection process or the post-detection process for the treatment tool is disabled. The photographic subjects of the category to which the detected photographic subject belongs include, in addition to a treatment tool, a lesion in a case where a specific lesion such as a tumor or a non-tumor is detected, an area in a case where a specific area such as the esophagus or the duodenal bulb is detected, and the like.

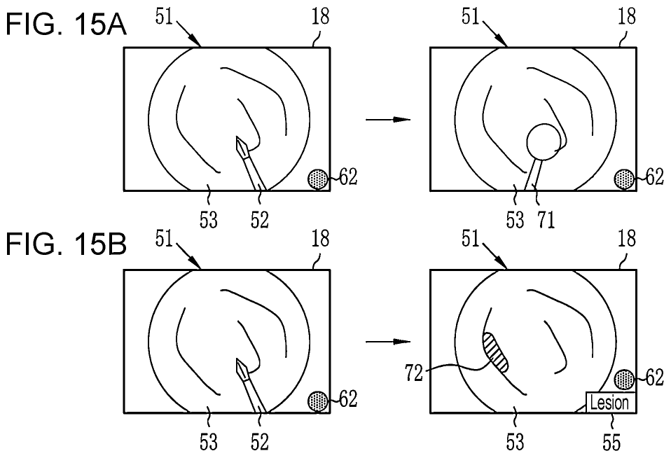

As illustrated in FIG. 15A, when the forceps 52 are erroneously detected as a snare, the detected photographic subject is the forceps 52, and the photographic subject of the type to which the forceps 52 belong is a treatment tool. Thus, a detection process or a post-detection process for the treatment tool is disabled in the disabled mode. Thus, in the disabled mode in this case, when the snare 71 is included in the endoscopic image 51, a detection process or a post-detection process for the snare 71 is not performed. Because the disabled mode in which the detection process or the like for a treatment tool is disabled continues, the disabled mode mark 62 is displayed on the display 18. A detection process for a treatment tool is performed by the treatment tool detector 45. Thus, the disabled mode in this case may be executed by not operating the treatment tool detector 45 among the various detectors.

As illustrated in FIG. 15B, in the disabled mode in which a detection process or a post-detection process for a treatment tool is disabled, when a detection process or a post-detection process for the forceps 52 is not performed and the endoscopic image 51 includes the lesion 72, a detection process or a post-detection process for the lesion 72 is performed in the detection process by the detection processing unit 12. In the detection process for the lesion 72, for example, “lesion” is generated as the detection result 54 and is displayed as the detection result indication 55 on the display 18. Because the disabled mode in which the detection process or the like for a treatment tool is disabled continues, the disabled mode mark 62 is displayed on the display 18.

In addition, for example, a photographic subject as a target of disabling may be a detected individual photographic subject. For example, when the forceps 52 are detected, the forceps 52 are regarded as a target of disabling, and forceps or the like other than the forceps 52 are not regarded as a target of disabling.

The photographic subject serving as a target of disabling need not necessarily match the contents of the detection process. For example, when the detection process is a process of detecting a photographic subject on a type-by-type basis, the photographic subject as a target of disabling can be set to the category to which the type of the photographic subject belongs, not the type of photographic subject. For example, when a snare is detected in a detection process of detecting a treatment tool on a type-by-type basis, the photographic subject as a target of disabling may be set to the category of the treatment tool to which the snare belongs.

The disabled mode in which a detection process or the like for a treatment tool or the like is disabled may be implemented by disabling various detection processes. Thus, in the disabled mode, any one or more of a lesion detection process or a post-detection process of the lesion detection process, a treatment tool detection process or a post-detection process of the treatment tool detection process, and an area detection process or a post-detection process of the area detection process may be disabled.

In the disabled mode, as a result of disabling a detection process or a post-detection process for a detected photographic subject, for example, even if erroneous detection is repeated in the detection process, it is possible to prevent a wrong photographic subject name from being continuously displayed or recorded due to the erroneous detection. By disabling only the post-detection process, even in the disabled mode, the detection process or the post-detection process can be continued depending on the photographic subject included in the endoscopic image 51. Thus, it is possible to prevent a wrong photographic subject name from being continuously displayed or recorded due to erroneous detection, and perform a notification process or a recording process in response to detection of another photographic subject. Thus, flexible setting makes it possible to, in the case of erroneous detection, omit the labor of various corrections due to display, recording, or the like of a wrong detection result 54 and effectively perform a detection process.

When the operation mode of the medical image processing apparatus 10 is the disabled mode and a preset cancellation condition is satisfied, the mode setting unit 15 cancels the disabled mode. Thus, the mode setting unit 15 sets the disabled mode in a period from when a specific instruction is provided from the user to when a specific cancellation condition is satisfied. The cancellation condition may be, for example, a time point at which a disabled mode duration time, which is a preset time, has elapsed from start of the disabled mode. The disabled mode duration time can be set as appropriate and is, for example, 3 minutes. After the disabled mode has been cancelled, the mode setting unit 15 sets the operation mode to an operation mode other than the disabled mode, for example, the operation mode immediately before the disabled mode is started. A setting may be made such that, after the disabled mode is cancelled, the operation mode shifts to a preset operation mode other than the operation mode before the shift to the disabled mode.

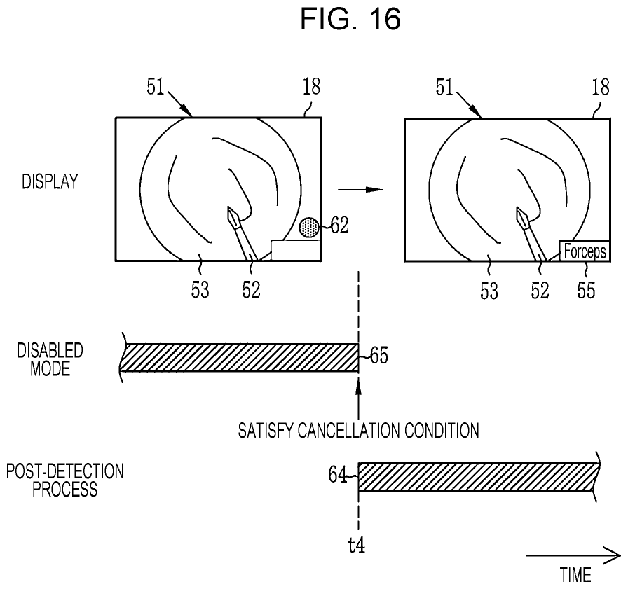

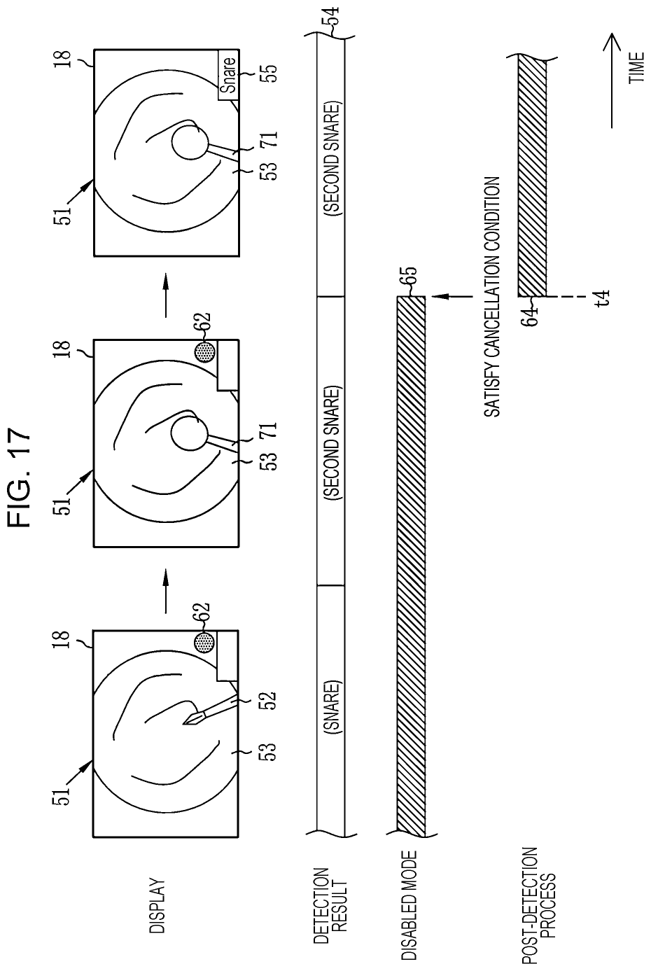

As illustrated in FIG. 16, in the disabled mode in which a detection process or a post-detection process for the forceps 52 as a detected photographic subject is disabled, the cancellation condition for the disabled mode is satisfied and the disabled mode is automatically cancelled after 3 minutes, set as a disabled mode duration time, have elapsed from the start of the disabled mode. Time t4 is the time at which 3 minutes have elapsed from the disabled mode start time.

The cancellation condition can be based on the endoscopic image 51. In this case, it is necessary to perform a detection process on the endoscopic image 51. Thus, only a post-detection process is disabled and a detection process is not disabled in the disabled mode. The cancellation condition is, for example, a case where a photographic subject different from the detected photographic subject is detected in the endoscopic image 51. The range of a photographic subject different from the detected photographic subject can be set in accordance with a process of detecting the photographic subject, and can be similar to the examples of a detected photographic subject and a photographic subject to be disabled as described above.

Thus, when the detected photographic subject is a detected individual photographic subject, the cancellation condition is satisfied and the disabled mode is cancelled in response to a photographic subject different from the detected individual photographic subject being detected in the endoscopic image 51. When the detection process is a process of detecting a photographic subject on a type-by-type basis, the cancellation condition is satisfied and the disabled mode is cancelled in response to a photographic subject of a type different from that of the photographic subject detected in the endoscopic image 51 being detected.

As illustrated in FIG. 17, in the disabled mode in which a post-detection process for the forceps 52 as a detected photographic subject is disabled, when the snare 71 is detected in the endoscopic image 51 after the disabled mode starts, a detection result 54 different from that in a case where the forceps 52 are erroneously detected is generated as a detection result because the photographic subject is different. For example, when an erroneous detection result 54 “snare” is generated for the forceps 52, the detection process recognizes that the forceps 52 and the snare 71 are different from each other in the shape or the like, and a detection result 54 “second snare” or the like is generated for the snare 71. When the cancellation condition is that a photographic subject different from the detected photographic subject has been detected and the detection result 54 is a “second snare” different from “snare”, the cancellation condition is satisfied, the disabled mode is cancelled at time t4 at which the cancellation condition is satisfied, the post-detection process disabled in the disabled mode is enabled, and individual processes set as the post-detection process start.

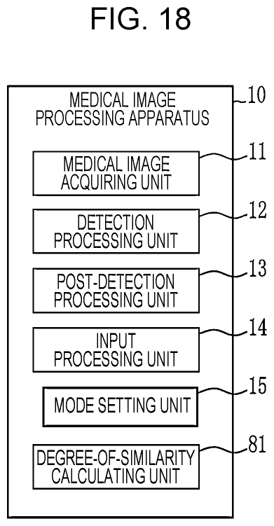

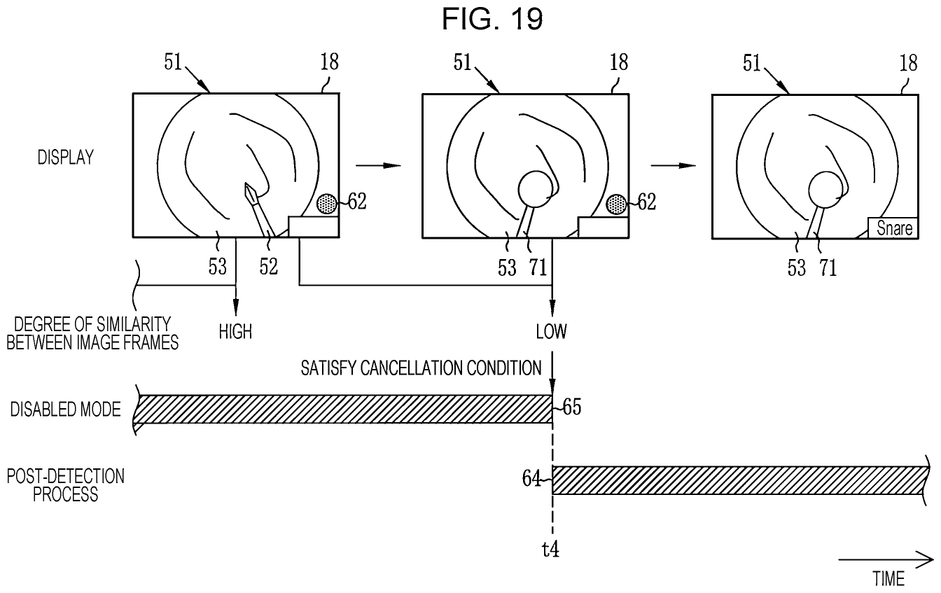

The cancellation condition may be a condition using a degree of similarity between a plurality of endoscopic images 51 acquired by the medical image acquiring unit 11. In this case, as illustrated in FIG. 18, the medical image processing apparatus 10 includes a degree-of-similarity calculating unit 81. The degree-of-similarity calculating unit 81 calculates a degree of similarity indicating the degree to which a plurality of endoscopic images are similar to each other. As a method for calculating a degree of similarity, various feature quantities can be calculated from endoscopic images, and a degree of similarity can be calculated by using the calculated feature quantities. The feature quantities may be feature vectors in the plurality of endoscopic images 51. A degree of similarity may be calculated based on the mutual proximity based on the feature vectors.

In the case of using a degree of similarity, the cancellation condition is that the degree of similarity between a plurality of most recently acquired endoscopic images 51 is lower than or equal to a preset value. The degree of similarity is the degree to which the plurality of endoscopic images 51 are similar to each other. When the degree of similarity is high, the degree to which the plurality of endoscopic images are similar to each other is high. When the degree of similarity is low, the degree to which the plurality of endoscopic images are similar to each other is low. The plurality of endoscopic images acquired most recently may be the endoscopic image acquired latest and endoscopic images of a preset number of frames acquired in the past. A frame is the unit of capturing of an endoscopic image. One endoscopic image is acquired per frame.

As illustrated in FIG. 19, in the present embodiment, the degree of similarity between two endoscopic images is calculated in the order of latest acquisition. When the calculated degree of similarity is lower than or equal to a preset threshold value, “low” is input to the field of “degree of similarity between image frames”. When the degree of similarity is high and is not lower than or equal to the threshold value, “high” is indicated similarly. When the degree of similarity is “high” and is not lower than or equal to the preset threshold value, the cancellation condition is not satisfied, and thus the disabled mode is continued without being cancelled. When the degree of similarity is “low”, which is lower than or equal to the threshold value, the cancellation condition is satisfied and the disabled mode is cancelled. After the disabled mode has been cancelled, the operation mode returns to the operation mode before the shift to the disabled mode.

The case in which a degree of similarity is used to cancel the disabled mode is preferable because the disabled mode can be automatically and appropriately cancelled even when an error in a detection process continues.

When the disabled mode is cancelled, it is preferable to notify the user. Upon the disabled mode being cancelled, the indicator 61 indicating the disabled mode is turned off. Thus, the notification indicating that the disabled mode has been cancelled may be common to the indication by the indicator 61 (FIG. 9). Accordingly, it is preferable that the medical image processing apparatus 10 notify the user in response to receiving a specific instruction from the user, in the case of setting the operation mode to the disabled mode, or in the case of cancelling the setting of the disabled mode.

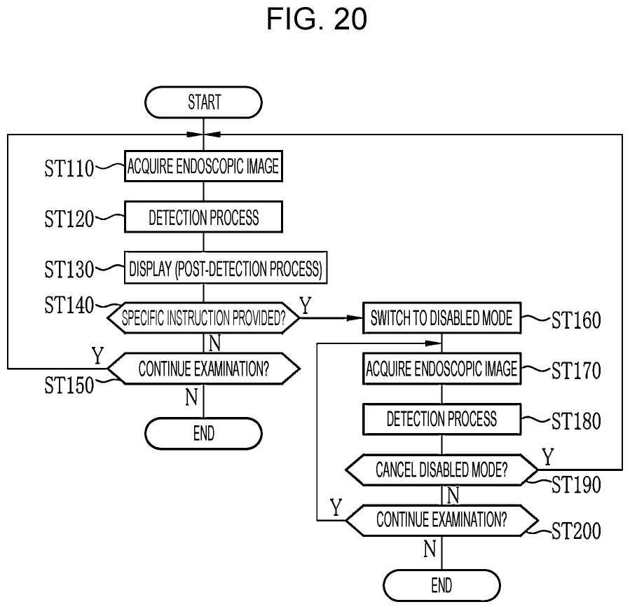

Next, a description will be given of the flow of a process performed by the medical image processing apparatus 10 according to the present embodiment. As illustrated in FIG. 20, the medical image acquiring unit 11 acquires the endoscopic image 51 acquired by the endoscope apparatus 16 (step ST110). The detection processing unit 12 performs a detection process on the endoscopic image 51 acquired by the medical image acquiring unit 11 (step ST120).

The detection result 54 obtained in the detection process is displayed as the detection result indication 55 on the display 18 (step ST130). The user determines whether the detection result 54 is an erroneous detection by viewing the detection result indication 55 and the endoscopic image 51 displayed on the display 18, and provides a specific instruction using a foot switch or the like, which is the input device 19, if the detection result 54 is an erroneous detection and the operation mode is to be switched to the disabled mode (Y in step ST140). If the specific instruction has been provided, the operation mode is switched to the disabled mode (step ST160).

After the operation mode has been switched to the disabled mode, in the disabled mode of the present embodiment, a detection process is performed and a post-detection process is not performed, and thus an endoscopic image is acquired (step ST170) and a detection process is performed (step ST180). If the disabled mode is to be cancelled (Y in step ST190), the disabled mode is cancelled, the operation mode returns to the operation mode immediately before the disabled mode is set, and acquisition of an endoscopic image is started (step ST110). If the disabled mode is not to be cancelled (N in step ST190) and if the examination is to be continued (Y in step ST200), an endoscopic image is acquired again and a detection process is performed in the disabled mode (steps ST170 and ST180). If the examination is not to be continued (N in step ST200), the examination ends.

If a specific instruction is not provided (N in step ST140) and if the examination is to be continued (Y in step ST150), an endoscopic image is acquired again and a detection process is performed (steps ST110 and ST120). If the examination is not to be continued (N in step ST150), the examination ends.

The above-described embodiment and so forth include a program for a medical image processing apparatus. The program causes a computer to execute a function of acquiring a plurality of time-series medical images; a function of performing a detection process of detecting a photographic subject from each medical image of the plurality of medical images; a function of performing, in response to the photographic subject being detected, a post-detection process whose details are predetermined in accordance with the detected photographic subject, the post-detection process including at least a process of providing a notification about the detected photographic subject to a user; and a function of setting, in response to a specific instruction being provided from the user after the notification has been provided, a disabled mode in which the post-detection process for at least the detected photographic subject is disabled in a preset period.

In the above-described embodiment, the hardware structure of a processing unit, such as the medical image acquiring unit 11, the detection processing unit 12, the post-detection processing unit 13, the input processing unit 14, the mode setting unit 15, and the degree-of-similarity calculating unit 81, included in the medical image processing apparatus 10 serving as a processor apparatus may be various types of processors described below. The various types of processors include a central processing unit (CPU), which is a general-purpose processor that executes software (program) and functions as various processing units; a programmable logic device (PLD), which is a processor whose circuit configuration is changeable after manufacturing, such as a field programmable gate array (FPGA); a dedicated electric circuit, which is a processor having a circuit configuration designed exclusively for executing various processing operations, and the like.

A single processing unit may be constituted by one of these various types of processors or may be constituted by a combination of two or more processors of the same type or different types (for example, a combination of a plurality of FPGAs or a combination of a CPU and an FPGA). A plurality of processing units may be constituted by a single processor. Examples of constituting a plurality of processing units by a single processor are as follows. First, as represented by a computer of a client or server, a single processor is constituted by a combination of one or more CPUs and software, and the processor functions as a plurality of processing units. Secondly, as represented by a system on chip (SoC) or the like, a processor in which a single integrated circuit (IC) chip implements the function of an entire system including a plurality of processing units is used. In this way, various types of processing units are constituted by using one or more of the above-described various types of processors as a hardware structure.

Furthermore, the hardware structure of the various types of processors is, more specifically, electric circuitry formed by combining circuit elements such as semiconductor elements.

### REFERENCE SIGNS LIST

10 medical image processing apparatus
11 medical image acquiring unit
12 detection processing unit
13 post-detection processing unit
14 input processing unit
15 mode setting unit
16 endoscope apparatus
17 PACS
18 display
19 input device
21 control unit
22 communication unit
23 storage unit
24 data bus
25 network
31 CPU
32 RAM
33 ROM
34 medical image processing apparatus program
35 medical image processing apparatus data
36 temporary storage unit
37 data recording unit
41 lesion detecting unit
42 treatment tool detecting unit
43 area detecting unit
44 lesion detector
45 treatment tool detector
46 area detector
51 endoscopic image
52 forceps
53 lumen wall
54 detection result
55 detection result indication
56 option indication
57 cancellation mechanism activation mark
61 indicator
62 disabled mode mark
63 input period
64 post-detection process period
65 disabled mode period
66 to 69 input
71 snare
72 lesion
81 degree-of-similarity calculating unit
t1 to t4 time
ST110 to ST200 step

What is claimed is:

1. A medical image processing apparatus comprising:
one or more processors configured to:
acquire a plurality of time-series medical images;
perform a detection process of detecting a photographic subject from each medical image of the plurality of medical images;
perform, in response to the photographic subject being detected, a post-detection process whose details are predetermined in accordance with the detected photographic subject, the post-detection process including at least a process of providing a notification about the detected photographic subject to a user; and
set, in response to a specific instruction being provided from the user after the notification has been provided, an operation mode to a disabled mode in which the post-detection process for at least the detected photographic subject is disabled in a preset disabled period.

2. The medical image processing apparatus according to claim 1, wherein the detection process is at least one of a lesion detection process of detecting a lesion, a treatment tool detection process of detecting a treatment tool, or an area detection process of detecting an area.

3. The medical image processing apparatus according to claim 2, wherein the lesion detection process is a process of detecting the lesion on a type-by-type basis, and the treatment tool detection process is a process of detecting the treatment tool on a type-by-type basis.

4. The medical image processing apparatus according to claim 1, wherein the post-detection process includes a process of recording a result of the detection process in a storage.

5. The medical image processing apparatus according to claim 1, wherein the post-detection process includes a process of displaying, on a display, an option predetermined in accordance with the detected photographic subject.

6. The medical image processing apparatus according to claim 1, wherein the one or more processors are configured to:
receive input from the user; and
determine, based on the input, whether the specific instruction has been provided.

7. The medical image processing apparatus according to claim 6, wherein the one or more processors are configured to determine that the specific instruction has been provided, in response to the input being continuously performed for a preset continuous-input determination period or more in an input reception period during which the input is received.

8. The medical image processing apparatus according to claim 6, wherein the one or more processors are configured to determine that the specific instruction has been provided, in response to the input being continuously performed for a preset continuous-input determination period or more in a period during which the post-detection process for the photographic subject is disabled.

9. The medical image processing apparatus according to claim 6, wherein the one or more processors are configured to determine that the specific instruction has been provided, in response to the input being performed at least once in an input reception period during which the input is received.

10. The medical image processing apparatus according to claim 7, wherein the input reception period is a period during which the post-detection process is performed.

11. The medical image processing apparatus according to claim 6, wherein the one or more processors are configured to determine that the specific instruction has been provided, in response to the input being performed a preset number of times or more in a preset multiple-input determination period.

12. The medical image processing apparatus according to claim 2, wherein the one or more processors are configured to disable, in the disabled mode, the post-detection process for the lesion and/or a plurality of types of the treatment tool.

13. The medical image processing apparatus according to claim 1, wherein the one or more processors are configured to, when the post-detection process includes a plurality of processes, disable, in the disabled mode, at least one of the processes included in the post-detection process.

14. The medical image processing apparatus according to claim 2, wherein the one or more processors are configured to, in response to detecting the legion, disable, in the disabled mode, the post-detection process related to the lesion detection process.

15. The medical image processing apparatus according to claim 2, wherein the one or more processors are configured to, in response to detecting the treatment tool, disable, in the disabled mode, the post-detection process related to the treatment tool detection process.

16. The medical image processing apparatus according to claim 2, wherein the one or more processors are configured to, in response to detecting the area, disable, in the disabled mode, the post-detection process related to the area detection process.

17. The medical image processing apparatus according to claim 1, wherein the one or more processors are configured to disable, in the disabled mode, the detection process for the detected photographic subject.

18. The medical image processing apparatus according to claim 2, wherein the one or more processors are configured to disable, in the disabled mode, the detection process for the lesion and/or a plurality of types of the treatment tool.

19. The medical image processing apparatus according to claim 17, wherein the one or more processors are configured to, when the detection process includes a plurality of processes, disable, in the disabled mode, at least one of the processes included in the detection process.

20. The medical image processing apparatus according to claim 1, wherein the one or more processors are configured to set the disabled period to be a period from a time point at which the specific instruction is provided to a time point at which a specific cancellation condition is satisfied.

21. The medical image processing apparatus according to claim 20, wherein the cancellation condition is that a photographic subject different from the detected photographic subject is detected in the medical image.

22. The medical image processing apparatus according to claim 20, wherein the one or more processors are configured to calculate a degree of similarity indicating a degree to which the plurality of medical images are similar to each other, and
the cancellation condition is that the degree of similarity between the plurality of medical images acquired most recently is lower than or equal to a preset value.

23. The medical image processing apparatus according to claim 1, wherein the processor is configured to provide a notification to the user in response to receiving the specific instruction from the user, in a case of setting the disabled mode, or in a case of cancelling setting of the disabled mode.

24. A method for operating a medical image processing apparatus, the method comprising:
a step of acquiring a plurality of time-series medical images;
a step of performing a detection process of detecting a photographic subject from each medical image of the plurality of medical images;
a step of performing, in response to the photographic subject being detected, a post-detection process whose details are predetermined in accordance with the detected photographic subject, the post-detection process including at least a process of providing a notification about the detected photographic subject to a user; and
a step of setting, in response to a specific instruction being provided from the user after the notification has been provided, an operation mode to a disabled mode in which the post-detection process for at least the detected photographic subject is disabled in a preset disabled period.

25. A non-transitory computer readable medium for storing a computer-executable program, the computer-executable program causing a computer to execute:
a function of acquiring a plurality of time-series medical images;
a function of performing a detection process of detecting a photographic subject from each medical image of the plurality of medical images;
a function of performing, in response to the photographic subject being detected, a post-detection process whose details are predetermined in accordance with the detected photographic subject, the post-detection process including at least a process of providing a notification about the detected photographic subject to a user; and
a function of setting, in response to a specific instruction being provided from the user after the notification has been provided, an operation mode to a disabled mode in which the post-detection process for at least the detected photographic subject is disabled in a preset disabled period.
